(19) United States
(12) Patent Application Publication
(10) Pub. No.: US 2006/0286105 A1
(43) Pub. Date: Dec. 21, 2006

(54) Tgf-beta antagonists combined with renin-angiotensin-aldosteron-system antagonists for treating renal insufficiency

(76) Inventors: Steven Ledbetter, Westborough, MA (US); Ariela Benigni, Bergamo (IT); Giuseppe Remuzzi, Bergamo (IT)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/553,595

(22) PCT Filed: Apr. 30, 2004
(86) PCT No.: PCT/US04/13677

§ 371 Date: Oct. 19, 2005

Related U.S. Application Data
(60) Provisional application No. 60/466,417, filed on Apr. 30, 2003.

(51) Int. Cl. A61K 39/395; A61K 31/401; A61K 38/17

(52) U.S. Cl. 424/145.1; 514/423; 514/12

(57) ABSTRACT

The disclosure provides methods for treating, preventing, and reducing risk of occurrence of renal insufficiency in mammals. The disclosed methods include administering to a subject susceptible to, or afflicted with, renal disorder therapeutically effective amounts of a TGF-β antagonist and a renin-angiotensin-aldosterone system (RAAS) antagonist so as to maintain desirable levels of renal function. The populations treated by the methods of the invention include but are not limited to patients suffering or at risk for the development of renal insufficiency, such as those afflicted with type I or type II diabetes, hypertension, (auto)immune disease, systemic fibrosis, etc.

This application claims priority to U.S. patent application Ser. No. 60/466,417, filed Apr. 30, 2003, herein incorporated by reference.

### FIELD OF THE INVENTION

The present invention relates to the field of clinical pathophysiology, and more particularly to methods for treating or preventing renal dysfunction by administering renin-angiotensin-aldosterone system antagonists and TGF-β antagonists.

### BACKGROUND OF THE INVENTION

The kidneys function to reabsorb water and to concentrate and to remove waste metabolites from the circulatory system. The kidneys also have a number of regulatory functions which include maintenance of the pH, salt balance, and volume of the blood as well as stimulation of erythrocyte production. Because of the vital functions the kidneys perform in maintaining proper fluid and homeostasis, loss of renal function represents a life-threatening event. Acute or chronic loss of kidney function, due to injury, disease, or some intrinsic disorder, can cause a variety of systemic complications. End stage renal failure is currently only treatable by dialysis or organ transplantation. Diabetic patients represent the largest group of patients with end stage renal disease. In the United States alone, about 6 to 8 million people are afflicted with diabetes. Approximately 30 percent of patients with type I diabetes and 10-40 percent of those with type II diabetes will eventually suffer renal failure.

The kidneys produce renin, the enzyme which plays a central role in the regulation of blood pressure and kidney function. Renin enzymatically cleaves angiotensinogen, produced by the liver, to release angiotensin (Ang) I, which is in turn converted to Ang II by angiotensin converting enzyme (ACE, EC 3.4.15.1). Ang-II , a potent vasoconstrictor, causes blood pressure to rise. Ang II also stimulates the adrenal cortex to release aldosterone, which causes the renal tubules to reclaim more sodium ions from the filtrate. Because fewer Ang II receptors are expressed on the afferent arterioles than on the efferent arterioles, Ang II causes the efferent arterioles to constrict to a greater extent, thereby increasing the glomerular hydrostatic pressure. Although the renin-angiotensin-aldosterone system (RAAS) contributes to renal autoregulation, its major role is to stabilize systemic blood pressure and extracellular fluid volume.

Abnormal activation of RAAS is a common event in patients with chronic renal disease. Systemic hypertension has been suggested to be both a central cause and consequence of chronic renal disease. Hypertension leads to an increase in hydraulic pressure within the glomerular capillaries. Glomerular hypertension has a number of adverse effects, including increased protein filtration, which promotes release of cytokines and growth factors by mesangial cells and downstream tubular epithelial cells. A partial loss of kidney function also spurs compensatory renal growth. Glomerular hypertrophy and hypertension combine to increase capillary wall tension, promoting endothelial cell activation and injury, further release of cytokines and growth factors, and recruitment of anti-inflammatory cells. These mediators stimulate processes such as apoptosis, causing loss of normal kidney cells and increased matrix production, which leads to glomerular and interstitial fibrosis. The fibrosis in the renal cortex impairs the blood supply, further exacerbating hypoxic injury to the renal medulla. As additional nephrons are damaged, this self-perpetuating cycle is repeated and amplified through multiple pathways, ultimately culminating in renal failure.

The advent of anti-angiotensin drugs, such as ACE inhibitors and Ang II receptor antagonists, marked a significant step forward in therapy of hypertension and chronic renal insufficiency. Both types of drugs have been shown to slow the loss of renal function and retard the development of fibrosis. Nonetheless, these drugs do not prevent the progression of renal disease completely. Indeed, ACE inhibition does not ameliorate glomerular size-selective abnormalities in patients with type II diabetes, thus allowing for increased passage of proteins into the tubular lumen (Ruggenenti et.al. (1999) Kid. Int., 55:984-994): Proteinuria is one of the most powerful predicators for progression to chronic renal failure (D'Amico et al. (2003) Kid. Int., 63:809-825). Thus, a need exists for additional therapies that complement RAAS antagonists.

Current research concerning progression and treatment of kidney disorders has focused primarily on glowing the development of fibrotic disease in the renal cortex. Transforming growth factor (TGF)-β has been a target in the treatment of progressive glomerular fibrosis. TGF-β is a group of genetically related, multifunctional cytokines that regulate diverse cellular activities. Extensive research has identified TGF-β as a key player in inducing synthesis and slowing breakdown of extracellular matrix proteins (e.g., fibronectin, collagens, and proteoglycans) in glomeruli, the processes leading to fibrotic glomerular diseases such as glomerular sclerosis. Administration of TGF-β antagonists has been shown to slow the progression of cortical fibrosis. Furthermore, therapeutic benefits: of anti-TGF-β antibodies in restoring renal function in general, and function of the renal medulla in particular, have been recently demonstrated (WO 01/66140).

Despite this progress, the interplay between TGF-β and RAAS and the precise role of TGF-β in the initial loss of renal function remain poorly understood. Although ACE inhibitors or TGF-β antagonists slow the progression of fibrosis when administered individually, no additional reduction in fibrosis was observed in an animal model of acute renal disease (UUO model) when both therapies were conducted concomitantly.

Recent studies have reported that antiangiotensin therapy reduces plasma TGF-β levels in renal transplant patients (Campistol et al. (1999) Kidney Int., 56:714-719), and in patients with diabetic nephropathy (Sharma et al. (1999) Am. J. Kidney Dis. 34:818-823), or hypertension (Laviades et al: (2000) Hypertension, 36:517-522). However, other studies have reported that antiangiotensin therapy results in elevated plasma rennin. Further experimental evidence indicates that renin upregulates TGF-β via an Ang II-independent mechanism. Accordingly, it has been suggested that use of ACE inhibitors or Ang II receptor antagonists may prevent a therapeutic reduction in TGF-β (WO 00/40227; p. 35). Progress in developing therapeutic methods that exploit the relationship between TGF-β and RAAS has been constrained, in part, by the unavailability of appropriate animal models that mimic significant aspects of human renal insufficiency.

Therefore, there is a need in the art to understand the ultimate causes of renal dysfunction and to develop new therapeutic methods for treating and preventing loss of renal function.

### SUMMARY OF THE INVENTION

It is one of the objects of the present invention to provide methods and compositions for treating or preventing renal disorders characterized by or associated with a risk of diminution of renal function. Additional objects of the invention will be set forth in part in the following description, and in part will be understood from the description.

The present invention is based, in part, on the discovery and demonstration that treatment of chronic renal insufficiency in diabetic animals by concomitant administration of an anti-TGF-β antibody and an ACE inhibitor is more effective in slowing the loss of renal function than individual treatments with either drug.

The present invention provides methods for treating, preventing, and reducing risk of occurrence of renal insufficiency. The invention further provides methods for improving renal function, such as, e.g., pressure filtration, selective reabsorption, tubular secretion, and systemic blood pressure regulation. The disclosed methods include administering to a mammalian subject susceptible to, or afflicted with, a renal disorder, therapeutically effective amounts of a TGF-β antagonist and a renin-angiotensin-aldosterone system (RAAS) antagonist so as to maintain desirable levels of renal function as assessed by systemic blood and glomerular blood pressure, proteinuria, serum creatinine, etc. The populations treated by the methods of the invention include but are not limited to patients suffering or at risk for the development of renal insufficiency, such as those afflicted with type I or type II diabetes, hypertension, (auto)immune disease, systemic fibrosis, etc.

Methods of administration and compositions used in the methods of the inventions are provided. In the disclosed methods, a TGF-β antagonist and a RAAS antagonist are administered concurrently or consecutively over overlapping or nonoverlapping intervals.

TGF-β antagonists, used in the methods of the present invention, include but are not limited to antibodies directed against one or more isoforms of TGF-β, antibodies directed against TGF-β receptors; soluble TGF-β receptors and fragments thereof; and TGF-β inhibiting sugars and proteoglycans. In some embodiments, the TGF-β antagonist is a monoclonal antibody or a fragment thereof that blocks TGF-β binding to its receptor. Nonlimiting illustrative embodiments include a non-human monoclonal anti-TGF-β antibody, e.g., mouse monoclonal antibody 1D11 (also known as 1D11.16, ATCC Deposit Designation No. HB 9849), a derivative thereof (e.g., a humanized antibody) and a fully human monoclonal anti-TGF-β1 antibody (e.g., CAT192 described in WO,00/66631) or a derivative thereof.

RAAS antagonists, used in thee methods of the invention, include but are not limited to renin inhibitors, angiotensin-converting enzyme (ACE) inhibitors, and Ang II receptor antagonists. In illustrative embodiments, the RAAS antagonist is selected from the group consisting of aliskiren, enalkiren, remikirem, benazeprilat, captopril, enalapril, lisinopril, perindopril, quinapril, ramipril, benazepril, trandolapril, fosinopril, moexipril, perindopril, losartan, valsartan, irbesartan, candesartan, telmisartan, tasosartan, eprosartan, spironolactone, and eplerenone.

It is to be understood that both the foregoing general description and the following detailed description are exemplary and explanatory only and are not restrictive of the invention as claimed.

### BRIEF DESCRIPTION OF THE FIGURES

FIGS. 1A and 1B demonstrate the effect of the anti-TGF-β antibodies 1D11 and CAT192 alone or in combination with an ACEi on proteinuria in diabetic rats.

FIG. 2 demonstrates the effect of the anti-TGF-β antibodies 1D11 and CAT192 alone or in combination with ACEi on blood pressure in diabetic rats.

FIG. 3 demonstrates the effect of the anti-TGF-β antibodies 1D11 and CAT192 alone or in combination with an ACEi on renal histology in diabetic rats as measured by the percentage of glomeruli with sclerotic changes.

FIG. 4 demonstrates the effect of the anti-TGF-β antibodies 1D11 and CAT192 alone or in combination with an ACEi on renal histology in diabetic rats as measured by tubular damage scores.

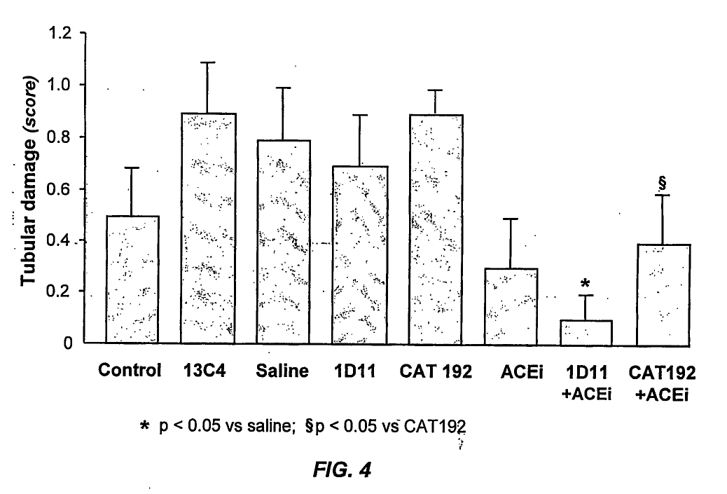

FIG. 5 demonstrates the effect of the anti-TGF-β antibody 1D11 alone or in combination with an ACEi on type III collagen deposition in the kidneys of diabetic rats.

FIG. 6 demonstrates the effect of the anti-TGF-β antibody 1D11 alone or in combination with an ACEi on renal interstitial infiltration of anti-inflammatory cells in kidneys of diabetic rats.

FIG. 7 shows proteinuria levels in diabetic rats treated in weeks 27-52 following induction of diabetes with (1) the irrelevant antibody 13C4, (2) the anti-TGF-β antibodies 1D11, or (3) enalapril.

FIG. 8 shows proteinuria levels in diabetic rats treated in weeks 52-61 following induction of diabetes with (1) the irrelevant antibody 13C4, (2) the anti-TGF-β antibodies 1D11, (3) enalapril, or (4) the combination of 1D11 and enalapril.

### DETAILED DESCRIPTION OF THE INVENTION

In order that the present invention may be more readily understood, certain terms are first defined. Additional definitions are set forth throughout the detailed description.

The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, humanized, human, single-chain, chimeric, synthetic, recombinant, hybrid, mutated, and CDR-grafted antibodies. The term “antigen-binding domain” refers to the part of an antibody molecule that comprises the area specifically binding to or complementary to a part or all of an antigen. Where an antigen is large, an antibody may only bind to a particular part of the antigen. The “epitope,” or “antigenic determinant” is a portion of an antigen molecule that is responsible for specific interactions with the antigen-binding domain of an antibody. An antigen-binding domain may comprise an antibody light chain variable region (V_{L}) and an antibody heavy chain variable region (V_{H}). An antigen-binding domain may be provided by one or more antibody variable domains (e.g., a so-called Fd antibody fragment consisting of a V_{H} domain or a so-called Fv antibody fragment consisting of a V_{H} domain and a V_{L} domain). The term “anti-TGF-β antibody,” or “antibody against at least one isoform of TGF-β,” refers to any antibody that specifically binds to at least one epitope of TGF-β. The terms “TGF-β receptor antibody” and “antibody against a TGF-β receptor” refer to any antibody that specifically binds to at least one epitope of a TGF-β receptor (e.g., type I, type II, or type III).

The terms “therapeutic compound” as used herein, refer to any compound capable of “antagonizing” TGF-β and/or RAAS by affecting biological activity of TGF-β and/or Ang II respectively, either directly or indirectly.

The term “renal function” refers to the ability of a kidney to perform its physiological functions such as pressure filtration, selective reabsorption, tubular secretion, and/or systemic blood pressure regulation. Methods for assessing renal function are well known in the art and include but are not limited to measurements of blood systemic and glomerular capillary pressure, proteinuria (e.g., albuminurea), microscopic and macroscopic hemaurea, serum creatinine level (e.g., one formula for estimating renal function in humans equates a creatinine level of 2.0 mg/dl to 50 percent of normal kidney function and 4.0. mg/dl to 25 percent), decline in the glomerular filtration rate (GFR) (e g., rate of creatinine clearance), and degree of tubular damage. Nonlimiting illustrative methods for assessing renal function are set forth in the Examples and in WO 01/66140.

The terms “inhibitor,” “inhibit,” “neutralize,” “antagonize,” and their cognates refer to the ability of a compound to act as an antagonist of a certain reaction or biological activity. The decrease in the amount or the biological activity is preferably at least about 10%, 20%, 30%, 40%, 50%, 60%, 70%, 80%, 90%, or more. The terms refer to a decrease in the relative amount of at least one protein that is responsible for the biological activity of interest (e.g., TGF-β, TGF-β receptor, Ang II, and Ang II receptor).

As used herein, “TGF-β antagonist” generally refers to any compound that directly downregulates the biological activity of TGF-β. A molecule “directly downregulates” the biological activity of TGF-β if it downregulates the activity by interacting with a TGF-β gene, a TGF-β transcript, a TGF-β ligand, or a TGF-β receptor. A TGF-β antagonist may, for example, bind to-and neutralize the activity of TGF-β; decrease TGF-β expression levels; affect stability or conversion of the precursor molecule to the active, mature form; interfere with the binding of TGF-β to one or more receptors; or it may interfere with intracellular signaling of a TGF-β receptor. Methods for assessing neutralizing biological activity of TGF-β antagonists are known in the art.

The term “renin-angiotensin-aldosterone system (RAAS) antagonist” refers to a compound having the ability to downregulate the amount or the biological activity of Ang II. The term is inclusive of renin inhibitors, angiotensin converting enzyme (ACE) inhibitors, Ang II receptor antagonist (also known as “Ang II receptor blockers”), and aldosterone antagonists. For a review of the mechanisms of action and clinical utilities of various RAAS antagonists see, for example, Physician's Desk Reference (PDR) 2003, 57th ed., Medical Economics Company, 2002.

The term “angiotensin-converting enzyme (ACE) inhibitor (ACEi)” refers to a compound having the ability to inhibit the cleavage of the N-terminal dicapeptide Ang I to the vasoactive octapeptide Ang II. For a review of various ACEi see, for example, Am. J. Cardiol., 66, 7D-13D (1990). The methods for assessing biological activity of ACEi are known in the art and can be performed, for example, using the method of Cushman (1971) Biochem. Pharm., 20:1637-1645 or as described in Wei et al. (1992) J. Biol. Chem.,267, 13398-13405.

The term “renin inhibitor” refers to a compound having the ability to inhibit the initial, rate-limiting step in the RAAS cascade, i.e., the renin-mediated, proteolytic conversion of angiotensinogen into the N-terminal decapeptide Ang 1, the penultimate precursor to Ang II. For a review of various assays for measuring renin amounts and its biological activity, see, e.g., Cartledge et al. (2000) Biochem., 37:262-278.

The terms “angiotensin (Ang) II receptor antagonist” and “Ang II receptor blocker” refer to a compound having the ability to inhibit the vasoactive effects of endogenous Ang II by competitive blockade at an Ang II receptor (e.g., type I (AT₁) and/or type II (AT₂)) located in vascular smooth muscle and within the adrenal gland. For a detailed review of Ang II receptors and the various antagonists thereof see, for example, Pharmacol. Rev., 45, 206-242 (1993). The biological activity of an Ang II receptor antagonist can be assessed using, for example, modifications of radioligand binding assay of Gunther et al. (1980) Circ. Res. 47:278.

The term “aldosterone antagonist” refers to a compound having the ability to counteract the effect of aldosterone, e.g., by competitive blockage aldosterone receptors found in renal tubules.

The terms “treatment,” “therapeutic method,” and their cognates refer to treatment or prophylactic/preventative measures. Those in need of treatment may include individuals already having a particular medical disorder as well as those who may ultimately acquire the disorder.

The terms “therapeutically effective dose,” or “therapeutically effective amount,” refer to that amount of a compound that results in prevention or delay off onset or amelioration of symptoms of renal dysfunction in a subject or an attainment of a desired biological outcome, such as improved renal function. The effective amount can be determined by methods well-known in the art and as described in the subsequent sections.

The terms “specific interaction,” or “specifically binds,” or their cognates, mean that two molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Kₐ is higher than 10⁶ M⁻¹, or preferably higher than 10⁸ M⁻¹. If necessary, nonspecific binding can be reduced without substantially affecting specific binding by varying the binding conditions. Such conditions are known in the art and a skilled artisan using routine techniques can select appropriate conditions. The conditions are usually defined in terms of concentration of antibodies, ionic strength of the solution, temperature, time allowed for binding, concentration of unrelated molecules (e.g., serum albumin, milk casein), etc.

The phrase “substantially identical” means that a relevant amino acid sequence is at least 70%,,75%, 80%, 85%, 90%, 95%, 97%, 98%, 99%, or 100% identical to a given sequence. By way of example, such sequences may be variants derived from various species, or they may be derived from the given sequence by truncation, deletion, amino acid substitution or addition. Percent identity between two amino acid sequences may be determined by standard alignment algorithms such as, for example, Basic Local Alignment, Tool (BLAST) described in Altschul et al. (1990) J. Mol. Biol., 215:403-410, the algorithm of Needleman et al. (1970) J. Mol. Biol., 48:444-453, or the algorithm of Meyers et al. (1988) Comput. Appl. Biosci., 4:11-17. Such algorithms are incorporated into the BLASTN, BLASTP, and “BLAST 2 Sequences” programs (see www.ncbi.nlm.nih.gov/BLAST). When utilizing such programs, the default parameters can be used. For example, for nucleotide sequences the following settings can be used for “BLAST 2 Sequences”: program BLASTN, reward for match 2, penalty for mismatch −2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter-ON.

As used herein, “TGF-β,” unless otherwise specifically indicated, refers to any one or more isoforms of TGF-β. Likewise, the term “TGF-β receptor,” unless otherwise indicated, refers to any receptor that binds at least one TGF-β isoform. Currently, there are 5 known isoforms of TGF-β (TGF-β1-β5), all of which are homologous among each other (60-80% identity), form homodimers of about 25 kDa, and act upon common TGF-β receptors (TβR-I, TβR-II, TβR-IIB, and TβR-III). TGF-β1, TGF-β2, and TGF-β3 are found in mammals. The structural and functional aspects of TGF-β as well as TGF-β receptors are well known in the art (see, for example, Cytokine Reference, eds. Oppenheim et al., Academic Press, San Diego, Calif., 2001). TGF-β is remarkably conserved among species. For example, the amino acid sequences of rat and human mature TFG-β1s are nearly identical. Thus, antagonists of TGF-β are expected to have a high species cross-reactivity.

Compositions and Methods

The present invention is based, in part, on the discovery and demonstration that treatment of chronic renal insufficiency in diabetic animals by concomitant administration of an anti-TGF-β antibody and an ACE inhibitor is more effective in slowing the loss of renal function than individual treatments with either drug.

The present invention provides methods for treating, preventing, and reducing risk of occurrence of renal insufficiency in mammals. The invention further provides methods for improving renal function, such as, e.g., pressure filtration, selective reabsorption, tubular secretion, and systemic blood pressure regulation. The disclosed methods comprise administering to a subject susceptible to, or afflicted with, a renal disorder, therapeutically effective amounts of a TGF-β antagonist and a RAAS antagonist.

TGF-β Antagonists

TGF-β is a disulfide linked dimer that is synthesized as a preproprotein of about 400 amino acids (aa) which is cleaved prior to secretion to produce mature TGF-β. The N-terminal cleavage fragment, known as the “latency-associated peptide” (LAP), may remain noncovalently bound to the dimer, thereby inactivating TGF-β. TGF-β, isolated in vivo, is found predominantly in this inactive “latent” form associated with LAP. Latent TGF-β complex may be activated in several ways, for example, by binding to cell surface receptors called the cation-independent mannose-6-phosphate/insulin-like growth factor II receptor. Binding occurs through mannose-6-phosphate residues attached at glycosylation sites within LAP. Upon binding to the receptor, TGF-β is released in its mature form. Mature, active TGF-β is then free to bind to its receptor and exert its biological functions. The major TGF-β-binding domain in the type II TGF-β receptor has been mapped to a 19 amino acid sequence (Demetriou et al. (1996) J. Biol. Chem., 271:12755).

Examples of TGF-β antagonists that may be used in the methods of the present invention include but are not limited to: monoclonal and polyclonal antibodies directed against one or more isoforms of TGF-β (U.S. Pat. No. 5,571,714; WO 97/13844; WO 00/66631; dominant negative and soluble TGF-β receptors or antibodies directed against TGF-β receptors (Flavell et al. (2002) Nat. Rev. Immunol., 2(1):46-53; U.S. Pat. No. 5,693,607; U.S. Pat. No.6,001,969; U.S. Pat. No. 6,008,011; U.S. Pat. No. 6,010,872; WO 92/00330; WO 93/09228; WO 95/10610; and WO 98/48024); LAP (WO 91/08291); LAP-associated TGF-β (WO 94/09812); TGF-β-binding glycoproteins/proteoglycans such as fetuin (U.S. Pat. No. 5,821,227); decorin, biglycan, fibromodulin, lumican, and endoglin (U.S. Pat. No. 5,583,103,; U.S. Pat. No. 5,654,270; U.S. Pat. No. 5,705,609; U.S. Pat. No. 5,726,149; U.S. Pat. No 5,824,655; U.S. Pat. No. 5,830,847; U.S. Pat. No. 6,015,693; WO 91/04748; WO 91/10727; WO 93/09800; and WO 94/110187); mannose-6-phosphate or mannose-1-phosphate (U.S. Pat. No. 5,520,926); prolactin (WO 97/40848); insulin-like growth factor II (WO98/17304); extracts of plants, fungi and bacteria (EU 813875; JP 8119984; and U.S. Pat. No. 5,693,610); antisense oligonucleotides (U.S. Pat. No. 5,683,988; U.S. Pat. No. 5,772,995; U.S. Pat. No. 5,821,234; U.S. Pat. No. 5,869,462; and WO 94/25588); proteins involved in TGF-β signaling, including SMADs and MADs (EP 874046; WO 97/31020; WO 97/38729; WO 98/03663, WO 98/07735; WO 98/07849; WO 98/45467; WO 98/53068; WO 98/55512; WO 98/56913; WO 98/53830; WO 99/50296; U.S. Pat. No. 5,834,248; U.S. Pat. No. 5,807,708; and U.S. Pat. No. 5,948,639), Ski and Sno (Vogel (1999) Science, 286:665; and Stroschein et al. (1999) Science, 286:771-774); and any mutants, fragments, or derivatives of the above-identified molecules that retain the ability to directly inhibit the biological activity of TGF-β.

In some embodiments, the TGF-β antagonist is an antibody that blocks TGF-β binding to its receptor. The antibody is such that it specifically binds to at least one isoform of TGF-β or to the extracellular domain of at least one TGF-β receptor. In some other embodiments, the anti-TGF-β antibody specifically binds at least one isoform of TGF-β selected from the group consisting of TGF-β1, TGF-β2, and TGF-β3. In yet other embodiments, the anti-TGF-β antibody specifically binds to at least: (a) TGF-β1, TGF-β2, and TGF-β3 (also referred to as “pan-neutralizing antibody”); (b) TGF-β1 and TGF-β2; (c) TGF-β1 and TGF-β3; and (d) TGF-β2 and TGF-β3. In various embodiments, the affinity constant Kₐ of the TGF-β antibody for at least one isoform of TGF-β, which it specifically binds, is preferably greater than 10⁸ M⁻¹, 1⁷ M⁻¹, 10⁸ M⁻¹, 10⁹ M⁻¹, 10¹⁰ M⁻¹, 10¹¹ M⁻¹, or 10¹² M⁻¹. In yet further embodiments, the antibody of the invention specifically binds to a protein substantially identical to human TGF-β, TGF-β, and/or TGF-β. Also contemplated for use in humans are humanized forms and derivatives of nonhuman antibodies derived from any vertebrate species described in the cited references. Producing such variants is well within the ordinary skill of an artisan (see, e.g., Antibody Engineering, ed. Borrebaeck, 2nd ed., Oxford University Press, 1995).

In nonlimiting illustrative embodiments, the anti-TGF-β antibody is a murine monoclonal antibody 1D11 produced by the hybridoma 1D11.16 (ATCC Deposit Designation No. 9849, also described in U.S. Pat. Nos. 5,571,714; 5,772,998; and 5,783,185). The sequence of the 1D11 heavy chain variable region is available under accession No. AAB46787. Thus, in related embodiments, the anti-TGF-β antibody is a derivative of 1D11, e.g., an antibody comprising the CDR sequence identical to those in AAB46787 such as a humanized antibody. In yet further nonlimiting illustrative embodiments, the anti-TGF-β antibody is a fully human recombinant antibody generated by phage display, such as CAT192 described in WO 00/66631, or an antibody comprising the CAT192 CDR sequences disclosed therein.

While the 1D11 antibody specifically binds all three mammalian isoforms of TGF-β, CAT192 specifically binds TGF-β only. The antigen affinities for 1D11 and CAT192 are approximately 1 nM and 8.4 pM, respectively. The epitopes for 1D11 (Dasch et al. (1998) J. Immunol., 142:1536-1541) and CAT192 have been mapped to the C-terminal portion of mature TGF-β.

RAAS Antagonists

In the methods of the present invention, one or more TGF-β antagonists are used in combination with one a more RAAS antagonists. The RAAS antagonist is an agent selected from the group consisting of a renin inhibitor, an ACE inhibitor, and an Ang II receptor antagonist.

Renin inhibitors used in the methods of the present invention include but are not limited to:

aliskiren (SPP100): 2(S),4(S),5(S),7(S)-N-(2-carbamoyl-2-methylpropyl)-5-amino-4-hydroxy-2,7-diisopropyl-8-[4-methoxy-3-(3-methoxypropoxy—)phenyl]-octanamid hemifumarate and compounds related thereto as disclosed in U.S. Pat. No. 5,719,141 and WO 01/09079;

enalkiren: [1S-(1R*,2S *,3R*)]-N-(3-amino-3-methyl-1-oxobutyl)-O-methyl-L-tyrosyl-N-[1-(cyclohexylmethyl)-2,3-dihydroxy-5-methylhexyl]-L-histidinamide and compound related thereto;

remikiren: (S)-2-tert-Butylsulphonylmethyl-N-[(S)-1-[(1S,2R,3S) -1-cyclo-hexylmethyl-3-cyclopropyl-2,3-dihydroxypropylcarbamoyl]-2-(1H-imidazol-4-yl)methyl]-3-phenlylpropionamide and compound related thereto;

and other renin inhibitors as described in U.S. Pat. Nos. 4,814,342; 4,855,303; 4,895,834, and 5,696,116.

ACE inhibitors used in the methods of the present invention include but are not limited to (examples of commercially available pharmaceutical formulations containing such compounds are given in parentheses):

benazepril (lotensin™, lotrel™): 3-[[1-(ethoxy-carbonyl)-3-phenyl-(1S)-propyl]amino]-2,3,4,5-tetrahydro-2-oxo-1H-1-(3S)-benzazepine-1-acetic acid monohydrochloride and its metabolite benazeprilat and compounds related thereto (U.S. Pat. No. 4,410,520);

captopril (capoten™): 1-[(2S)-3-mercapto-2-methylpropionyl]-L-proline and compounds related thereto (U.S. Pat. No. 4,105,776);

enalapril (vasotec™): 1-[N-[(S)-1-carboxy-3-phenylpropyl]-L-alanyl]-L-proline-1′-ethyl ester;

lisinopril (zestril™, privinil™): 1-[N₂-[(S)-1-carboxy-3-phenylpropyl]-L-lysyl]-L-proline and the various carboxyalkyl dipeptide derivatives and compounds related thereto (U.S. Pat. Nos. 4,374,829, 6,468,976, and 6,465,615);

perindopril erbumine (aceon™, coversyl™): (2S,3αS,7αS)-1-[(S)-N-[(S)-1-Carboxy-butyl]alanyl]hexahydro-2-indolinecarboxylic acid, 1-ethyl ester and compounds related thereto;

quinapril (accupril™): (3S)-2-[N-[(S)-1-ethoxycarbonyl-3-phenylpropyl]-L-alanyl]-1,2,3,4-tetrahydro-isoquinoline-3-carboxylique monochlorhydrate and compounds related thereto;

ramipril (altace™): (2S,3αS,6αS)-1[(S)-N-[(S)-1-carboxy-3-phenylpropyl]alanyl]octahydrocyclopenta[β]pyrrole-2-carboxylic acid, 1-ethyl ester and compounds related thereto;

trandolapril (mavik™): (2S,3αR,7αS)-1-[(S)-N-[(S)-Carboxy-3-phenylpropyl]alanyl]hexahydro-2-indolinecarboxylic acid, 1-ethyl ester and compounds related thereto;

fosinopril (monopril™): L-proline, 4-cyclohexyl-1-[[[2-methyl-1-(1-oxopropoxy)propoxyl](4-phenylbutyl) phosphinyl]acetyl] sodium salt, trans—, and compounds related thereto;

moexipril (univasc™): 3S-[2[R*(R*)],3R*]]-2-[2-[[1-(ethoxycarbonyl)-3-phenylpropyl]amino]-1-oxopropyl]-1,2,3,4-tetrahydro-6,7-dimethoxy-3-isoquinolinecarboxylic acid monohydrochloride and compounds related thereto; and

imidapril (tanatril™): (−)-4S)-3-[(2S)-2-[[(1S)-1-ethoxycarbonyl-3-phenylpropyl]amino]-propionyl]-1-methyl-2-oxoimidazolidine-4-carboxylic acid hydrochloride and compounds related thereto.

Additional examples of ACE inhibitors include those described in U.S. Pat. Nos. 5,696,116; 6,410,524, and 6,482,797.

Ang II receptor antagonists used in the methods of the present invention include but are not limited to:

losartan (cozaar™): 2-butyl-4-chloro-1-[p-(o-1H-tetrazol-5-ylphenyl)benzyl]imidazole-5-methanol, monopotassium salt and the various substituted imidazole derivatives and other compounds related thereto (U.S. Pat. No. 5,138,069);

valsartan, (diovan™): N-[p-(o-1H-tetrazol-5-yl-phenyl)benzyl]-N-valeryl-L-valine and compounds related thereto (U.S. Pat. No. 5,399,578);

irbesartan (avapro™): 2-n-butyl-4-spirocyclopentane-1-((2′-tetrazol-5-yl)biphenyl4-yl)-2-imidazolin-5-one and compounds related thereto (U.S. Pat. Nos. 5,270,317and 5,352,788);

candesartan (amias™, atacand™): 1-(cyclohexyloxycarbo-nyloxy)ethyl-2-ethoxy-1-[[2′-(1H-tetrazol-5-yl)biphenyl-4-yl]methyl]benzimidazole-7-carboxylate and compounds related thereto (in U.S. Pat. No. 5,196;444);

telmisartan (micardis™): 4′-[(1,4′-dimethyl-2′-propyl[2,6-bi-1H-benzimidazol]-1-yl)methyl]-[1,1′-biphenyl]-2-carboxylic acid and compounds related thereto (European Pat. Application No.6502314);

tasosartan (verdia™): 5,8-dihydro-2,4-dimethyl-8-[p-(o-1H-tetrazol-5-ylphenyl]pyrido[2,3-d]pyrimidin-7(6H)-one and compounds related thereto (U.S. Pat. No. 5,149,699);

eprosartan (teveten™): (E)-2-butyl-1-(p-carboxybenzyl)-α-2-thenylimidazole-5-acrylic acid and compounds related thereto (U.S. Pat. No. 5,185,351); and

saralasin: 1-(N-methylglycine)-5-L-valine-8-L-alanineangiotensin II (an octapeptide analog of Ang II (bovine) with amino acids 1 and 8 replaced with sarcosine and alanine, respectively.

Additional examples of suitable Ang II receptor antagonists include those described in U.S. Pat. Nos. 5,484,780; 6,028,091; and 6,329,384.

Aldosterone antagonists used in the methods of the present invention include but are not limited to:

eplerenone (inspra™): (7α, 11α, 17α)-pregn-4-ene-7,21-dicarboxylic acid,9,11-epoxy-17-hydroxy-3-oxo-,γ-lactone, methyl ester and compounds related thereto (U.S. Pat. No. 4,559,332); and

spironolactone (aldactone™): 7α-acetylthio-3-oxo-17α-pregn-4-ene-21,17-carbolactone and compounds related thereto.

Additional examples of suitable aldosterone antagonists include those described in U.S. Pat. No. 6,410,524.

Pharmaceutically acceptable salts of compounds disclosed herein can be used. Pharmaceutically acceptable salts include but are not limited to salts formed with metals, e.g., sodium, calcium, potassium, zinc, and magnesium, or with acids, e.g., sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, 2-butyne-1,4 dioate, 3-hexyne-2,5-dioate, benzoate, chlorobenzoate, hydroxybenzoate, methoxybenzoate, phthalate, xylenesulfonate, phenylacetate, phenylpropionate, phenylbutyrate, citrate, lactate, hippurate, B-hydroxybutyrate, glycollate, maleate, tartrate, methanesulfonate, propanesulfonate, naphthalene-1-sulfonate, naphthalene-2-sulfonate, and mandelate.

The use of RAAS antagonists in combination with certain other therapeutic agents is well known. Such compounds include but are not limited to, β-receptor blockers, calcium channel blockers, sodium channel inhibitors, diuretics, aldosterone antagonists (U.S. Pat. No. 6,410,524), endothelin-1 antagonists (U.S. Pat. No. 6,329,384).

The methods of the invention comprise administering to a subject susceptible to, or afflicted with, renal disorder a therapeutically effective amount of a TGF-β antagonist and a therapeutically effective amount of a RAAS antagonist so as to maintain desirable levels of renal function as assessed by systemic blood and glomerular blood pressure, proteinuria, medullary blood flow, and/or medullary ischemic injury. The populations treated by the methods of the invention include but are not limited to patients suffering or at risk for the development of renal insufficiency.

The terms “renal disorder,” “renal insufficiency” and their cognates refer to a disease or condition that involves a loss of renal function. Such disorders include but are not limited to any acute or chronic disease or disorder that compromises renal circulation, causes tubular injury, or otherwise causes a diminution in renal function. A wide variety of diseases or disorders may include renal pathologies, including rheumatic/immunologic disorders, genetic/metabolic disorders, hematologic/oncologic disorders, infectious disorders, radiation injury, renal surgery, lithotripsy, or drug- or toxin-induced/nephrotoxic disorders. Such diseases or disorders include, but are not limited to, diabetic (type I and type II) nephropathy, radiational nephropathy, obstructive nephropathy, diffuse systemic sclerosis, pulmonary fibrosis, allograft rejection, hereditary renal disease (e.g., polycystic kidney disease, medullary sponge kidney, horseshoe kidney), glomerulonephritis, nephrosclerosis, nephrocalcinosis, systemic lupus erythematosus, Sjogren's syndrome, Berger's disease, systemic or glomerular hypertension, tubulointerstitial nephropathy, renal tubular acidosis, renal tuberculosis, and renal infarction. For a detailed review of renal disorders, see The Kidney: Physiology and Pathophysiology, eds. Seldin et al., 3rd ed., Lippincott Williams & Wilkins Publishers, 2000.

In general, patients with systemic hypertension are at increased risk for developing renal insufficiency. Thus, for humans, the patient groups that exhibit blood pressure (systolic or diastolic) higher than 130/80 or 140/90 mm Hg would benefit from the concomitant administration of a TGF-β antagonist and a RAAS antagonist.

Normally, less than 0.15 g of protein is excreted into the urine per 24 hour period. Almost all types of kidney disease cause mild (up to 500 mg per day) to moderate (up to 4 g per day) protein leakage into the urine. The normal concentration of albumin in the urine is less than 1.0 mg/dl. Generally, 5-30 mg/dl urinary albumin is considered microalbuminuria, and 30 mg/dl and up is considered macroalbuminuria. The normal values of serum creatinine are 0.6-1.5 mg/dl for men and 0.6-1.1 mg/dl for women. The relationship between creatinine levels, renal function, and the stage of renal disease is shown in Table 1.

TABLE 1

| Creatinine | Estimated reduction | |
|---|---|---|
| level (mg/dl) | of renal function | Stage of renal disease |
| 0.6-1.5 | Up to 25% | Reduced or diminished |
| | | renal reserve |
| >1.5 | >50% | Renal insufficiency |
| 4.8 | 75% | Renal failure |
| 10 | 90% | End-stage renal disease |

The methods of the invention may be particularly useful in patients with renal insufficiency, renal failure, or end-stage renal disease. For example, the methods of the invention can be used to treat, slow or reverse the progression of renal disease in patients whose renal function is below normal by 25%, 40%, 50%, 60%, 75%, 80%, 90% or more. In some embodiments, the invention provides a method of improving renal function that allows the patient's renal function to improve by at least 10%, 20%, 30%, 40%, 50%, 60%, 70%, 80%, 90%, 100% or more. Furthermore the treatment may be useful in patients with microalbuminuria, macroalbuminuria, and/or proteinuria levels of over 1, 2, 3, 4, 5, 6, 7, 8, 9, or 10 g or more per a 24 hour period, and/or serum creatinine levels of about 1.0, 1.5, 2.0, 2.5, 3, 3.5, 4.0, 4.5, 5, 5.5, 6, 7, 8, 9, 10 mg/dl or higher. In some embodiments, the invention provides a method of improving renal function that allows the patient's serum creatinine levels, proteinuria, and/or urinary albumin excretion to be reduced by at least 10%, 20%, 30%, 40%, 50%, 60%, 70%, or more. Other indications include but are not limited to type I and II diabetes, renal transplant, creatinine clearance levels of lower than 97 (men) and 88 (women) ml/min, blood urea of 20-25 mg/dl or higher, and/or as indicated by renal imaging (e.g., MRI, ultrasound), or renal biopsy.

Methods of administration and compositions used in the methods of the inventions are provided. In the disclosed methods, a TGF-β antagonist and a RAAS antagonist are administered concurrently or consecutively over overlapping or nonoverlapping intervals.

“Administration” is not limited to any particular delivery system and may include, without limitation, parenteral (including subcutaneous, intravenous, intramedullary, intraarticular, intramuscular, or intraperitoneal injection) rectal, topical, transdermal, or oral for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition (described earlier). Physiologically acceptable salt forms and standard pharmaceutical formulation techniques and excipients are well known to persons skilled in the art (see, e.g., Physician's Desk Reference (PDR) 2003, 57th ed., Medical Economics Company, 2002, and Remington: The Science and Practice of Pharmacy, eds. Gennado et al., 20th ed, Lippincott, Williams & Wilkins, 2000).

Administration of an antagonist to an individual may also be accomplished by means of gene therapy, wherein a nucleic acid sequence encoding the antagonist is administered to the patient in vivo or to cells in vitro, which are then introduced into a patient, and the antagonist (e.g., antisense RNA, soluble TGF-β receptor) is produced by expression of the product encoded by the nucleic acid sequence. Methods for gene therapy to deliver TGF-β antagonists are known to those of skill in the art (see, e.g., Fakhrai et al. (1996) Proc. Nat. Acad. Sci. U.S.A., 93:2909-2914).

A TGF-β antagonist and a RAAS antagonist are administered concurrently or consecutively over overlapping or nonoverlapping intervals. In the sequential administration, the TGF-β antagonist and the RAAS antagonist can be administered in any order. In some embodiments, the length of an overlapping interval is more than 2, 4, 6, 12, 24, or 48 weeks.

The antagonists are administered as the sole active compound or in combination with another compound or composition. Unless otherwise indicated, the antagonists is administered a dose of approximately from 1 μg/kg to 25 mg/kg, depending on the severity of the symptoms and the progression of the disease. Most commonly, antibodies are administered in an outpatient setting by weekly administration at about 0.1-10 mg/kg doses by slow intravenous (IV) infusion. The appropriate therapeutically effective dose of an antagonist is selected by a treating, clinician and would range approximately from 1 μg/kg to 20 mg/kg, from 1 μg/kg to 10 mg/kg, from 1 μg/kg to 1 mg/kg, from 10 μg/kg, to 1 mg/kg, from 10 μg/kg to 100 μg/kg, from 100 μg to 1 mg/kg, and from 500 μg/kg to 5 mg/kg. Additionally, specific dosages indicated in the Examples or in the Physician's Desk Reference (PDR) 2003, 57th ed., Medical Economics Company, 2002, may be used.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Treatment of Renally Compromised Rats with TGF-β Antagonists and/or ACE Inhibitors

Male Sprague-Dawley rats with initial body weights of 250 to 300 g were used in this study. Animal care and treatment were conducted in accordance with the U.S. National Research guidelines, 1996. All animals were housed in a room in which the temperature was kept constant on a 12-hour dark/12-hour light cycle and allowed free access to standard diet containing 20% protein by weight and tap water.

The animals were subjected to unilateral nephrectomy under anaesthesia one week prior to the induction of diabetes to accelerate the onset of renal disease. The animals were rendered diabetic by a single intravenous (i.v.) injection of streptozotocin (STZ, Zanosar, Upjohn, Kalamazoo, Mich.; 60 mg/kg body weight). Food and water consumption, weight gain and the blood glucose levels were monitored to determine the degree of diabetes. The blood was obtained from the tip of the tails and analyzed by a modified glucose dehydrogenase method. The rats were considered diabetic if the blood glucose concentration was above 20 mmol/l or if they drank at least 100 ml/day/ Blood glucose levels were maintained between 200 and 400 mg/dl throughout the study by daily subcutaneous injections of insulin.

Four months, after the induction of diabetes, animals were divided into seven groups of eight (unless otherwise indicated), which were treated for the next four months as follows:

group 1 received i.p. injections of the control antibody 13C4 (anti-verotoxin murine monoclonal lgG1 antibody, Genzyme, Framingham, Mass.) three times a week at a dose of 0.5 mg/kg.

group 2 received i.p. injections of 1D11 (murine monoclonal anti-TGF-β antibody, Genzyme) three times a week at a dose of 0.5 mg/kg;

group 3 received i.p. injections of 1D11 3 times a week at a dose of 0.5 mg/kg and was given 12.5 mg/l lisinopril in the drinking water;

group 4 received saline injections twice a week;

group 5received i.p. injections of CAT192 (human anti-TGF-β antibody, Genzyme) three times a week at a dose of 0.5 mg/kg;

group 6 received i.p. injections of CAT 192 3 times a week at a dose of 0.5 mg/kg and was given 12.5 mg/l lisinopril in the drinking water; and

group 7 was given 12.5 mg/l the ACE inhibitor lisinopril in the drinking water.

### Example 2

### Effect of TGF-β Antagonists and/or ACE Inhibitors on Proteinuria

This example illustrates the effect of anti-TGF-β antibodies 1D11 or CAT192 alone or in combination with an ACEi on proteinuria in diabetic rats.

Animals were treated as described in Example 1. At the end of the study, twenty-four hour samples were collected using metabolic cages and proteinuria was determined by a modified Coomassie blue G dye-binding assay for proteins with bovine serum albumin as standard as described in Reed et al. (1981), Anal. Biochem. 116:53-64. Mean values (±SEM) were calculated. The significance of differences in mean values among treated groups was analyzed using an analysis of variance followed by a Duncan's multiple-range test.

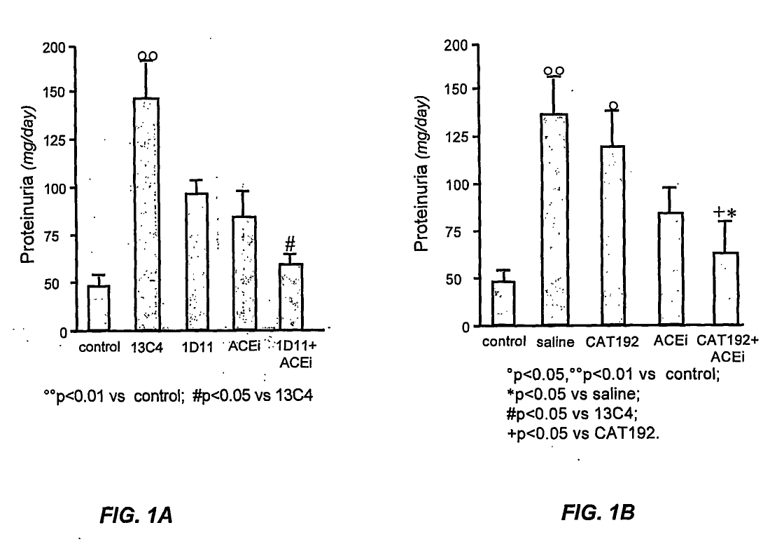

The results are presented in FIGS. 1A and 1B. The results demonstrate that proteinuria was reduced in groups treated with either 1D11 alone or in combination with lisinopril and that treatment with CAT192 in combination with lisinopril also reduced proteinuria as compared to control groups.

### Example 3

### Effect of TGF-β Antagonists and/or ACE Inhibitors on Blood Pressure

This example illustrates the effect of anti-TGF-β antibody alone or in combination with an ACEi on blood pressure in diabetic rats.

Animals were treated as described in Example 1. Systolic blood pressure at the end of the treatment period was recorded by tail plethysmography in conscious rats as described in Pfeffer et al. (1971) J. Lab. Clin. Med., 78:957-962.

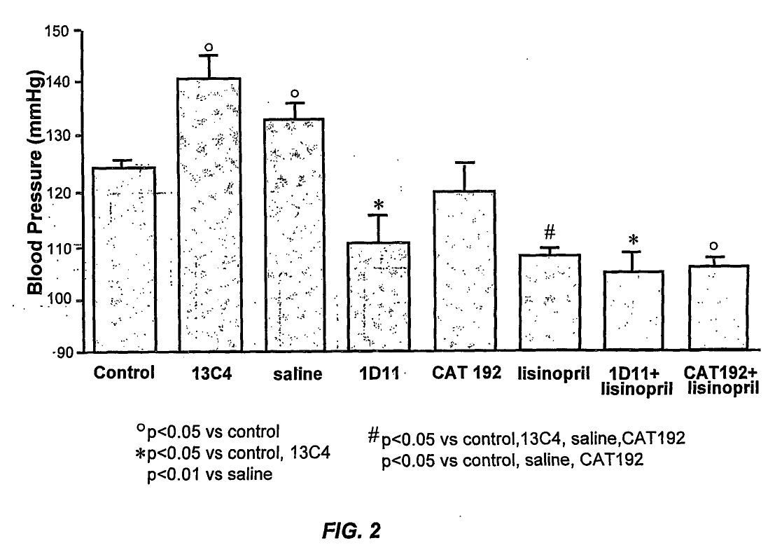

As shown in FIG. 2, both 1D11 and CAT192 displayed an anti-hypertensive effect. Lisinopril limited blood pressure increase to a similar extent as 1D11. Therefore, combination of either antibody with lisinopril fully controlled systolic blood pressure.

### Example 4

### Effect of TGF-β Antagonists and/or ACE Inhibitors on Glomerular Sclerosis

This example illustrates the effect of anti-TGF-β antibody alone or in combination with an ACEi on renal histology in diabetic rats as measured by the percentage of glomeruli with sclerotic changes.

Animals were treated as described in Example 1. At the end of the study, rat kidneys were harvested and fixed with 5% buffered formalin solution, embedded in paraffin, sectioned and stained with periodic acid-Schiff (PAS) for light microscopy analyses.

Glomerular diameters were measured and the degree of matrix expansion and glomerular injury was assessed on a minimum of 100 glomeruli/section. The degree of glomerulosclerosis was scored as previously described (Raij et al. (1984) Kidney Int., 26:137-143). The percentage of glomerular sclerosis was determined by an unbiased, trained pathologist who recorded pathologic changes using a 0-4 scale (score of 0 indicates no damage; 1+ indicates changes affecting less than 25% of the samples; 2+ indicates changes affecting 25- 50% of the sample; 3+, changes affecting 50-75% of the sample; 4+ changes affecting 75-100% of the sample).

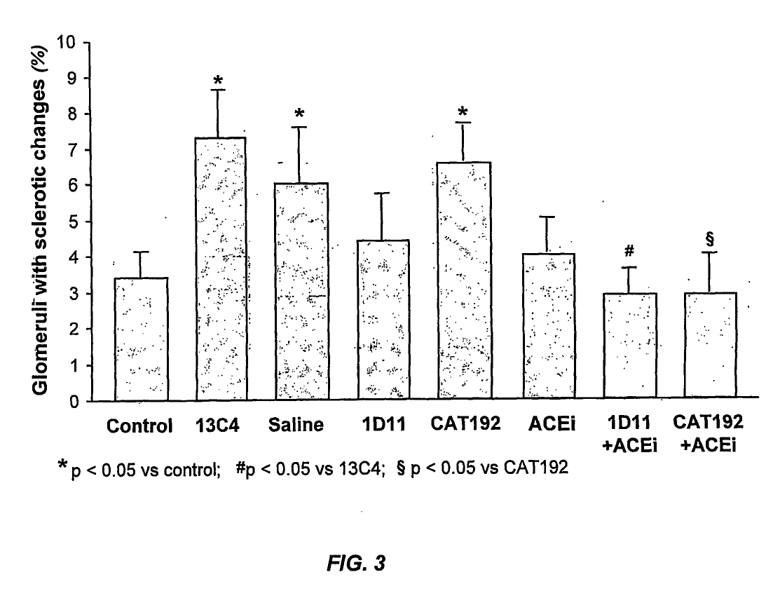

Evaluation of glomerular sclerosis is reported in FIG. 3. A variable degree of glomerular sclerosis and hyalinosis with segmental collapse of the glomerular tuft was detected in diabetic rats given control antibody or saline affecting on average 7.3. and 6.0% of the glomerular population. Administration of 1D11 led to a significant reduction in the percentage of glomeruli with sclerotic changes. Lisinopril limited the percentage of glomeruli with sclerosis to a similar degree as 1D 11. Therefore, complete renal protection was achieved by the combination therapies when compared to age-matched controls.

### Example 5

### Effect of TGF-β Antagonists and/or ACE Inhibitors on Tubular Integrity

This example shows the effect of anti-TGF-β antibodies 1D11 or CAT192 alone or in combination with an ACEi on renal histology in diabetic rats as measured by tubular damage scores.

Animals were treated as described in Examples 1 and 5. Histological sections were also examined (light microscopic sections stained with PAS) for fibrosis of vasa recta capillaries and the degree of tubulointerstitial damage. Tubular pathology was scored as described for glomerular sclerosis. The results indicate that combination therapy significantly reduced tubular damage to a level at or below control animals.

### Example 6

### Effect of TGF-β Antagonists and/or ACE Inhibitors on Renal Fibrosis

This example illustrates the effect of an anti-TGF-β antibody alone or in combination with an ACEi on type III collagen deposition in the kidneys of diabetic rats.

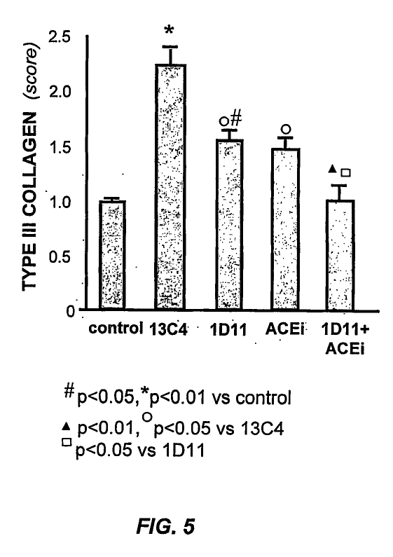

Animals were treated as described in Example 1 and 5. Type III collagen accumulation was detected by immunoperoxidase using a polyclonal rabbit anti-rat type III collagen antibody (Chemicon, Temecular, Calif.). Negative control was obtained by omitting the primary antibody. The signal intensity was graded on a scale of 0 to 3 (0, no staining; 1, weak staining; 2, moderate intensity; 3, strong staining. The results are illustrated in FIG. 5. The results indicate that animals treated with 1D11 in combination with lisinopril had reduced interstitial type III collagen deposition as compared to animals treated with the antibody or lisinopril alone. Under the same conditions, animals treated with CAT192 in combination with lisinopril had slightly lower (not statistically significant type III collagen deposition compared to animals treated with the antibody alone.

### Example 7

### Effect of TGF-β Antagonists and/or ACE Inhibitors on Renal Inflammation

This example illustrates the effect of an anti-TGF-β antibody alone or in combination with an ACEi on renal interstitial infiltration of anti-inflammatory cells in the kidneys of diabetic rats.

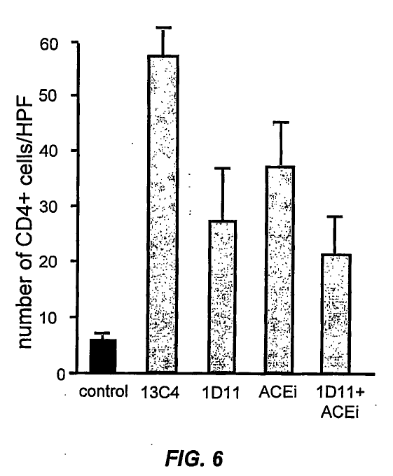

Animals were treated as described in Example 1 and 5. A mouse monoclonal antibody was used for detection of CD4+ helper T cells, thymocytes and macrophages. Staining was analyzed by indirect immunogluoresence technique. Positive cells were counted in at least 10 randomly selected high power microscopic fields (×400) per each animal. The results are illustrated in FIG. 6.

The results indicate that animals treated with 1D11 in combination with lisinopril had reduced interstitial anti-inflammatory cell infiltration as compared to animals treated with the antibody or lisinopril alone. Under the same conditions, animals treated with CAT192 in combination with lisinopril had slightly lower macrophage infiltration as compared to animals treated with the antibody alone.

### Example 8

### TGF-β Antagonists or ACE Inhibitors Individually Slow the Progression of Early Stage Diabetic Nephropathy

The goal of this study was to compare the antiproteinuric effect of 1D11 versus enalapril when treatment is started in the early phase (at 27 weeks post streptozotocin injection) of diabetic nephropathy.

Male Sprague-Dawley rats with initial body weight of 250-275 g were studied. All animals were allowed free access to standard diet and tap water. Diabetes was induced by a single intravenous injection of streptozotocin (60 mg/kg body weight). The presence of diabetes was confirmed 2 days later by the measurement of the tail blood glucose level with a reflectance meter. Diabetic rats received daily evening injections of insulin in doses individually adjusted to maintain a blood glucose level between 200 and 450 mg/dl. Blood glucose levels were monitored at least once a week in all diabetic rats and occasionally in control animals for comparison.

Diabetic rats were divided into 3 groups (n=6 each): group 1 received a control antibody, 13C4; groups 2 and 3 received 1D11 (0.5 mg/kg i.p. three times a week) or enalapril (15 mg/l in the drinking water), respectively from 27 to 52 weeks; group 4 was normal rats.

Systolic blood pressure was measured every two months after diabetic induction by the tail cuff method.

Urinary excretion of proteins was measured in 24 hr urine samples collected in metabolic cages at baseline, 18, 27, 36, 45, and 52 weeks by the modified Coomassie blue G dye-binding assay for proteins.

Urinary albumin excretion was determined at 52 weeks by ELISA with a specific antibody against rat albumin.

Serum creatinine was measured at the end of the study by autoanalyzer (CX5 Beckman).

Renal morphology was assessed as glomerulosclerosis, interstitial inflammation and tubular damage. Specifically, the removed kidneys were fixed overnight in Dubosq-Brazil, dehydrated in alcohol, and embedded in paraffin. Kidney samples were sectioned at 3-μm intervals, and the sections were stained with Masson's trichrome, hematoxylin and eosin, or periodic-acid Schiff reagent (PAS stain). Tubular (atrophy, casts, and dilation) and interstitial changes (fibrosis and inflammation) were graded from 0 to 4+ (0, no changes; 1+, changes affecting <25% of the sample; 2+, changes affecting 25 to 50% of the sample; 3+, changes affecting 50 to 75% of the sample; 4+, changes affecting 75 to 100% of the sample). At least 100 glomeruli were examined for each animal, and the extent of glomerular damage was expressed as the percentage of glomeruli presenting sclerotic lesions. All renal biopsies were analyzed by the same pathologist who was unaware of the nature of the experimental groups.

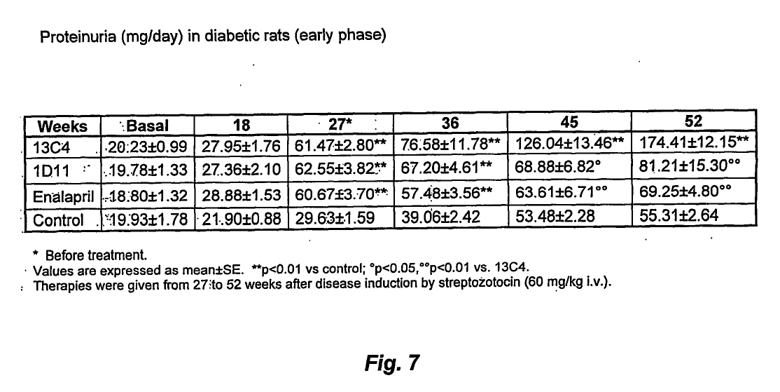

The statistical analysis was performed using the nonparametric Kruskal-Wallis test for multiple comparisons. The statistical significance level was defined as p<0.05.The results of the study are shown in FIG. 7 and Tables 2-5. The results indicate that, when dosed therapeutically in early stage diabetic nephropathy, 1D11 and enalapril are similarly efficacious in reducing proteinuria (FIG. 7), urinary albumin excretion (Table 2), renal histology (Table 3), systolic blood pressure (Table 4), and serum creatinine (Table 5).

TABLE 2
Urinary albumin excretion (mg/day) in
diabetic rats (early phase treatment)

| Group | 52 weeks |
|---|---|
| 13C4 | 29.14 ± 2.39* |
| 1D11 | 10.00 ± 0.71*° |
| enalapril | 4.70 ± 0.83*°# |
| Control | 1.72 ± 0.30 |

Values are expressed as mean ± SE.
*p < 0.05 vs control;
°p < 0.05 vs. 13C4;
#p < 0.05 vs 1D11.
Therapies were given from 27 to 52 weeks after disease induction by streptozotocin (60 mg/kg i.v.).

TABLE 3
Renal histology (52 weeks) in diabetic rats (early phase treatment)

| | Glomeruli with | Interstitial | |
|---|---|---|---|
| | sclerotic | inflammation | Tubular damage |
| Group | changes (%) | (score) | (score) |
| 13C4 | 4.16 ± 1.19** | 0.60 ± 0.20 | 0.73 ± 0.18 |
| 1D11 | 1.45 ± 0.40° | 0.17 ± 0.17 | 0.33 ± 0.21 |
| Enalapril | 1.53 ± 0.32° | 0.33 ± 0.21 | 0.50 ± 0.22 |
| Control | 0.92 ± 0.36 | 0 | 0.17 ± 0.17 |

Values are expressed as mean ± SE.
**p < 0.01 vs. control;
°p < 0.05 vs. 13C4.
Therapies were given from 27 to 52 weeks after disease induction by streptozotocin (60 mg/kg i.v.).

TABLE 4
Systolic blood pressure (mmHg) in diabetic rats (early phase treatment)

| | Weeks | | | |
|---|---|---|---|---|
| | 27* | 36 | 45 | 52 |
| 13C4 | 145.00 ± 2.24** | 157.50 ± 2.81** | 159.17 ± 3.96** | 161.67 ± 1.67** |
| 1D11 | 143.33 ± 2.47** | 135.83 ± 1.54**°° | 120.83 ± 3.27°° | 120.00 ± 1.83°° |
| Enalapril | 148.33 ± 2.47** | 127.50 ± 2.81**°° | 118.33 ± 2.47°° | 117.50 ± 2.14°° |
| Control | 108.33 ± 1.67 | 113.33 ± 1.67 | 109.17 ± 3.52 | 113.33 ± 4.77 |

*Before treatment.
Values are expressed as mean ± SE.
**p < 0.01 vs. control;
°°p < 0.01 vs. 13C4.
Therapies were given from 27 to 52 weeks after disease induction by streptozotocin (60 mg/kg i.v.).

TABLE 5
Serum creatinine (mg/dl) in diabetic rats (early phase treatment)

| Groups | 52 weeks |
|---|---|
| 13C4 | 0.51 ± 0.02 |
| 1D11 | 0.51 ± 0.02 |
| Enalapril | 0.49 ± 0.07 |
| Control | 0.49 ± 0.01 |

Values are expressed as mean ± SE.
Therapies were given from 27 to 52 weeks after disease induction by streptozotocin (60 mg/kg i.v.).

### Example 9

### TGF-β Antagonists in Combination with ACE Inhibitors Reverse Late Stage Diabetic Nephropathy

The goal of this study was to examine the effect of late intervention (started at 52 weeks after streptozotocin injection) with 1D11 and enalapril, alone or in combination, on the progression of late-stage diabetic nephropathy.

Diabetic rats were kept as described in Example 8 and were divided into 4 groups (n=5-6 each) and given from 52 to 61 weeks after the diabetis induction the following: the irrelevant antibody 13C4; the anti-TGF-β antibody 1D11 (0.5 mg/kg i.p. three times a week); the ACE inhibitor enalapril (15 mg/l in the drinking water), or the combination of 1D11 and enalapril. Five normal rats were used as controls. Systolic blood pressure was measured every two months after diabetes induction. Urinary excretion of proteins was measured in 24 hour urine samples collected in metabolic cages at baseline, 18, 27, 36, 45, 52, and 61 weeks. All measurements and data analysis were performed as described in Example 8.

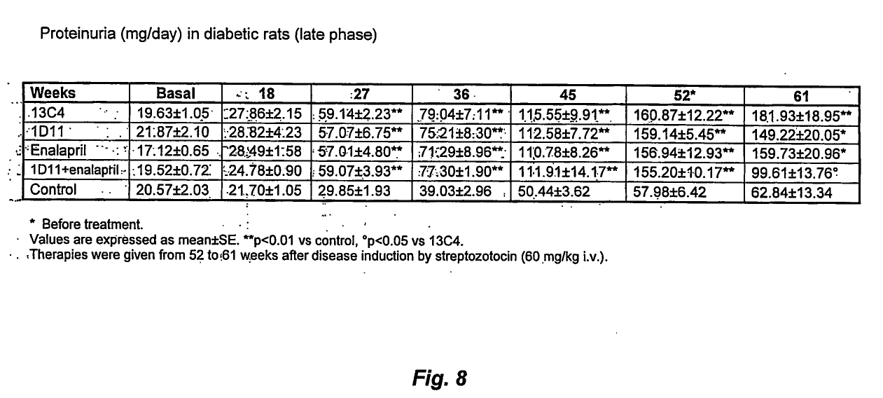

The results of the study are shown in FIG. 8 and Tables 6-9. The results indicate that, when dosed therapeutically in late stage diabetic nephropathy, 1D11,and enalapril are similarly efficacious in reducing proteinuria (FIG. 8), urinary albumin excretion (Table 6), renal histology (Table 7), systolic blood pressure (Table 8), and serum creatinine (Table 9). While both agents are efficacious, a combination therapy is significantly more efficacious, with reductions in some parameters approaching control values. While both agents reduce blood pressure, their combination is not more effective in this parameter. This suggests that the efficacy of the combination therapy is unlikely to be due to the hemodynamic effects of these agents.

TABLE 6
Urinary albumin excretion (mg/day) in
diabetic rats (late phase treatment)

| Groups | 61 weeks |
|---|---|
| 13C4 | 47.16 ± 9.77* |
| 1D11 | 28.30 ± 19.88* |
| enalapril | 30.40 ± 15.08* |
| 1D11 + enalapril | 7.27 ± 2.88° |
| Control | 3.49 ± 1.44 |

Values are expressed as mean ± SE.
*p < 0.05 vs control;
°p < 0.05 vs. 13C4.
Therapies were given from 52 to 61 weeks after disease induction by streptozotocin (60 mg/kg i.v.).

TABLE 7
Renal histology (61 weeks) in diabetic rats (late phase treatment)

| | Glomeruli with | Interstitial | Tubular |
|---|---|---|---|
| | sclerotic | inflammation | damage |
| Groups | changes (%) | (score) | (score) |
| 13C4 | 7.10 ± 1.91* | 0.75 ± 0.19 | 1.00 ± 0.00 |
| 1D11 | 3.33 ± 1.03 | 0.50 ± 0.29 | 0.57 ± 0.25 |
| enalapril | 4.60 ± 2.19 | 0.60 ± 0.24 | 0.60 ± 0.24 |
| 1D11 + | 1.80 ± 0.37° | 0° | 0.40 ± 0.24 |
| enalapril | | | |
| Control | 1.40 ± 0.24 | 0.40 ± 0.24 | 0.40 ± 0.24 |

Values are expressed as mean ± SE.
**p < 0.05 vs. control;
°p < 0.05 vs. 13C4.
Therapies were given from 52 to 61 weeks after disease induction by streptozotocin (60 mg/kg i.v.).

TABLE 8
Systolic blood pressure (mmHg) in diabetic
rats (late phase treatment)

| | Weeks | | |
|---|---|---|---|
| | 36 | 52 | 61 |
| 13C4 | 156.00 ± 1.00** | 162.00 ± 2.55** | 169.00 ± 2.45** |
| 1D11 | 154.17 ± 2.71** | 166.67 ± 3.57** | 131.00 ± 1.87*°° |
| Enalapril | 158.33 ± 2.79** | 165.00 ± 2.89** | 135.00 ± 1.58**°° |
| 1D11 + | 158.33 ± 3.80** | 165.83 ± 2.01** | 131.00 ± 2.92**°° |
| enalapril | | | |
| Control | 113.00 ± 2.00 | 116.00 ± 4.85 | 115.00 ± 3.54 |

*Before treatment.
Values are expressed as mean ± SE.
*p < 0.05,
**p < 0.01 vs. control;
°°p < 0.01 vs. 13C4.
Therapies were given from 52 to 61 weeks after disease induction by streptozotocin (60 mg/kg i.v.).

TABLE 9
Serum creatinine (mg/dl) in diabetic rats (late phase treatment)

| Groups | 61 weeks |
|---|---|
| 13C4 | 0.52 ± 0.02 |
| 1D11 | 0.55 ± 0.01 |
| Enalapril | 0.56 ± 0.07 |
| 1D11 + enalapril | 0.53 ± 0.03 |
| Control | 0.49 ± 0.01 |

Values are expressed as mean ± SE.
Therapies were given from 52 to 61 weeks after disease induction by streptozotocin (60 mg/kg i.v.).

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. The citation of any references herein is as not an admission that such references are prior art to the present invention. The representations of molecular mechanisms and pathways are provided for ease of the understanding of the invention only and should not be considered binding.

1. A method of treating a mammal having diminished renal function, comprising administering to the mammal a therapeutically effective amount of a TGF-β antagonist and a therapeutically effective amount of a RAAS antagonist in the amounts and for a period of time sufficient to treat renal insufficiency.

2. A method of slowing loss of renal function in a mammal having a renal disorder, comprising administering to the mammal a therapeutically effective amount of a TGF-β antagonist and a therapeutically effective amount of a RAAS antagonist thereby slowing the loss of the renal function.

3. The method of claim 2, wherein the renal function the loss of which is slowed is selected from the group consisting of pressure filtration, selective reabsorption, tubular secretion, and systemic blood pressure regulation.

4. The method of claim 1 or 2, wherein the RAAS antagonist is an ACE inhibitor.

5. The method of claim 4, wherein the ACE inhibitor is lisinopril.

6. The method of claim 1 or 2, wherein the TGF-β antagonist is selected from the group consisting of an anti-TGF-β antibody, an anti-TGF-β receptor antibody, and soluble TGF-β receptor.

7. The method of claim 8, wherein the anti-TGF-β antibody or the anti-TGF-β receptor antibody is human or humanized.

8. The method of claim 8, wherein the anti-TGF-β antibody specifically binds to TGF-β1, TGF-β2, and TGF-β3.

9. The method of claim 8, wherein the anti-TGF-β antibody specifically binds to TGF-β1 and TGF-β2.

10. The method of claim 8, wherein the antibody is 1D11 or a derivative thereof.

11. The method of claim 8, wherein the antibody specifically binds to TGF-β1.

12. The method of claim 11, wherein the antibody is CAT192 or a derivative thereof.

13. The method of claim 1 or 2, wherein the mammal is human.

14. The method of claim 1 or 2, wherein the mammal is diabetic.

15. The method of claim 1 or 2, wherein the mammal is hypertensive.

16. The method of claim 1 or 2, wherein the TGF-β antagonist and the RAAS antagonists are administered concomitantly for more than 2 weeks.

17. A method of improving renal function in a mammal having diminished renal function, the method comprising administering to the mammal a therapeutically effective amount of a TGF-β antagonist and a therapeutically effective amount of a RAAS antagonist to the mammal in the amounts and for a time period sufficient to improve the renal function.

18. The method of claim 17, the renal function is improved by at least 10%.

19. The method of claim 17, wherein the mammal has renal insufficiency.

20. The method of claim 17, wherein the mammal has renal failure.

21. The method of claim 17, herein the mammal has end-stage renal disease.

22. The method of claim 17, wherein the mammal is diabetic.

23. The method of claim 17, wherein the renal function which is improved is selected from the group consisting of pressure filtration, selective reabsorption, and tubular secretion.

24. The method of claim 17, wherein proteinuria is reduced by at least 10%.

25. The method of claim 17, wherein urinary albumin excretion is reduced by at least 10%.

26. The method of claim 17, wherein the RAAS antagonist is an ACE inhibitor.

27. The method of claim 17, wherein the ACE inhibitor is enalapril.

28. The method of claim 17, wherein the TGF-β antagonist is selected from the group consisting of an anti-TGF-β antibody, an anti-TGF-β receptor antibody, and soluble TGF-β receptor.

29. The method of claim 28, wherein the anti-TGF-β antibody or the anti-TGF-β receptor antibody is human or humanized.

30. The method of claim 28, wherein the anti-TGF-β antibody specifically binds to TGF-β1, TGF-β2, and TGF-β3.

31. The method of claim 28, wherein the anti-TGF-β antibody specifically binds to TGF-β1 and TGF-β2

32. The method of claim 28, wherein the TGF-β antibody is 1D11 or a humanized or human derivative thereof.

33. The method of claim 28, wherein the TGF-β antibody specifically binds to TGF-β1.

34. The method of claim 28, wherein the TGF-β antibody is CAT192 or a derivative thereof.

35. The method of claim 17, wherein the mammal is human.

36. The method of claim 17, wherein the mammal is diabetic.

37. The method of claim 17, wherein the mammal is hypertensive.

38. The method of claim 17, wherein the TGF-β antagonist and the RAAS antagonists are administered concomitantly for more than 2 weeks.
